(19) United States
(12) Patent Application Publication
(10) Pub. No.: US 2026/0069113 A1
(43) Pub. Date: Mar. 12, 2026

(54) DEVICE, SYSTEMS, AND METHODS FOR CREATING A WORKING SPACE WITHIN A BODY

(71) Applicant: BOSTON SCIENTIFIC SCIMED, INC., Maple Grove, MN (US)

(72) Inventors: Mauro Viquez Gonzalez, Heredia (CR); Marco Vinicio Arias Alpizar, Curridabat (CR); Gonzalo Jose Saenz Villalobos, Alajuela (CR); Oscar Segura Elizondo, San Jose (CR); Fernando Valerio Guerrero, Alajuela (CR); Viviana Artavia Salas, Heredia (CR); Erick Van Patten Rivera, San Jose (CR); Gustavo Carvajal Bonilla, Heredia (CR); Valeria Zúñiga Vargas, Heredia (CR); Rafael Berenzon, Cartago (CR); Gabriela Gamboa Bastos, San Jose (CR); Esteban de la Trinidad Jiménez Sánchez, San Jose (CR); Emma Cristina Quesada Matamoros, Alajuela (CR)

(73) Assignee: BOSTON SCIENTIFIC SCIMED, INC., Maple Grove, MN (US)

(21) Appl. No.: 19/320,485

(22) Filed: Sep. 5, 2025

Related U.S. Application Data
(60) Provisional application No. 63/691,632, filed on Sep. 6, 2024.

(51) Int. Cl. A61B 1/00; A61B 1/005; A61B 1/018; A61B 17/00

(52) U.S. Cl. CPC A61B 1/00147; A61B 1/00131; A61B 1/005; A61B 1/018; A61B 17/00234; A61B 2017/00296; A61B 2017/00367; A61B 2017/00862

(57) ABSTRACT

Devices, systems, and methods configured to form a chamber along or adjacent a target area within a patient at or along which a procedure is to be performed. The chamber is sealed with respect to the target area to form a sealed enclosed working area sealed from external anatomical structures, materials, substances, processes, etc. The target area is thus isolated so that a procedure may be limited to the working area without influence or interference of surrounding anatomical structures, materials, substances, processes etc.

### CROSS-REFERENCE TO RELATED APPLICATIONS

This application claims the benefit of priority under 35 U.S.C. § 119 to U.S. Provisional Application No. 63/691,632, filed Sep. 6, 2024, the entire disclosure of which is hereby incorporated by reference herein for all purposes.

### FIELD

The present disclosure relates generally to the field of devices, systems, and methods for creating a working space within the body of a patient during a medical and/or surgical procedure. More particularly, the present disclosure relates to medical devices, systems, and methods for creating a working space in communication with a target area along an anatomical structure, and isolating, such as fluidly isolating or sealing, the working space from surrounding areas such as anatomical structures or other materials or substances outside the working space.

### BACKGROUND

Various medical procedures are performed by creating a working space clear of interference by extraneous materials, such as other anatomical structures or substances not to be affected by the procedure. Various challenges are often presented by anatomical structures or materials or processes which may encounter or impede or otherwise interact with the working space, and which may interfere with performance of the procedure. Procedures may be performed in areas of the anatomy which continuously function during the process, and/or which must be cleared of materials normally present during usual functioning. Such functions or materials may interact and potentially interfere with the procedure. For instance, when a procedure is to be performed in the intestines, the bowel must be cleared prior to the procedure for the medical professional to be able to view the target site for the procedure and/or to eliminate potential interference by bowel contents. A significant issue prior to the start of any endoscopic gastrointestinal procedure involving the bowel is adequate preparation in the form of cleaning/cleansing of the bowel wall through a series of dietary restrictions and/or medication(s) to eliminate materials (e.g., fecal materials) from within the bowel. An inadequate bowel preparation can lead to, in the best case, a partial occlusion of the bowel wall, and, in the worst condition, a completely covered wall with a partially filled bowel. These conditions usually lead to an incomplete endoscopic procedure, or an altogether rescheduling of the procedure. This can lead to complications and an inefficient use of doctor resources. Solutions to these and other challenges in the art would be welcome.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a medical device is configured to create a working space along a target area within a body of a patient. In some aspects, the medical device includes a chamber device including a chamber member with a proximal end and a distal end and defining a chamber therein between the proximal end and the distal end and defining a working space in communication with the target area; and at least one sealing member operatively associated with the chamber to seal the working space from surrounding patient anatomy, materials, and/or substances. In some aspects, the at least one sealing member includes a proximal sealing member sealing the proximal end of the chamber member, and a distal sealing member sealing the distal end of the chamber member.

In some aspects, the chamber device includes a chamber wall defining the chamber and sized to fit within a body lumen within a patient to contact the interior of the body lumen; the proximal sealing member seals the proximal end of the chamber member with respect to the body lumen; the distal sealing member seals the distal end of the chamber member with respect to the body lumen; and at least one opening is defined through the chamber wall to communicate the chamber with the target area while the working space remains sealed from surrounding patient anatomy, materials, and/or substances.

In some aspects, the chamber device includes a chamber wall defining the chamber and formed of a plurality of elongate elements, and at least two adjacent elongate elements are spaced apart to define an opening through the chamber wall to communicate the chamber with the target area. In some aspects, the elongate elements are flexible to conform an exterior of the chamber wall to the target area with the at least one sealing member sealing against the target area. In some aspects, the elongate elements are flexible elongate elements are shiftable from a compact delivery configuration to an expanded configuration defining the chamber. In some aspects, the device further includes an elongate member having a distal end and a proximal end, the chamber device extending from a distal end of the elongate member; and a control handle operatively coupled with the plurality of elongate elements to shift the plurality of elongate elements between the delivery configuration and the expanded configuration. In some aspects, the at least one sealing member covers spaces between adjacent elongate elements to form a barrier between the chamber and the exterior of the chamber device.

In some aspects, the at least one sealing member comprises a membrane.

In some aspects, the at least one sealing member includes a sealing member which is flexible to allow a medical device to be passed between the flexible sealing member and the target area and to maintain a seal with respect to the target area.

In accordance with various principles of the present disclosure, a system is configured for creating a working space along a target area within a body of a patient. In some aspects, the system includes a chamber device including a chamber member with a proximal end and a distal end and defining a chamber therein between the proximal end and the distal end and in communication with the target area; and at least one sealing member forming a barrier sealing the chamber with respect to the target area to create a working space sealed from surrounding patient anatomy, materials, and/or substances; and the medical device has a distal end configured for use in performing a procedure with respect to the target area.

In some aspects, the medical device includes a delivery device with a lumen therethrough and an additional medical device extendable through the lumen. In some aspects, the medical device is a medical scope.

In some aspects, the medical device is extendable along the at least one sealing member and into the chamber.

In some aspects, the at least one sealing member includes a proximal sealing member sealed around the exterior of a distal end of the medical device with the distal end of the medical device extending into the chamber.

In accordance with various principles of the present disclosure, a method of creating a working space along a target area within a body of a patient includes advancing a chamber device to the target area, the chamber device defining a chamber; sealing a sealing member provided over the chamber device with respect to the target area to seal the chamber with respect to the target area and from surrounding patient anatomy, materials, and/or substances; and communicating the chamber with the target area via one or more openings defined through the chamber device and sealed with respect to the target area and from surrounding patient anatomy, materials, and/or substances.

In some aspects, the method further includes sealing a proximal sealing member provided over a proximal end of the chamber device with respect to the target area to seal a proximal end of the chamber with respect to the target area and from proximal surrounding patient anatomy, materials, and/or substances; and sealing a distal sealing member provided over a distal end of the chamber device with respect to the target area to seal a distal end of the chamber with respect to the target area and from distal surrounding patient anatomy, materials, and/or substances.

In some aspects, the method further includes advancing the chamber device into a body lumen and sealing the chamber device with respect to the body lumen to seal the chamber with respect to portions of the body lumen proximal and distal to the target area. In some aspects, the method further includes extending a distal end of the medical device past a proximal sealing member and into the chamber.

In some aspects, the medical device is a medical scope and a proximal sealing member seals about the exterior of a distal end of the medical scope to seal the chamber with respect to the distal end of the medical scope, the method further including advancing a further medical device through a working channel of the medical scope and into the chamber defined by the chamber device.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a system for creating a working space within a body of a patient and formed in accordance with aspects of the present disclosure.

FIG. 2 illustrates a proximal view of a device for creating a working space within a body of a patient such as illustrated in FIG. 1.

FIG. 3 illustrates a system, such as illustrated in FIG. 1, in use within a body lumen of a patient.

FIG. 4 illustrates a further use position of a system as illustrated in FIG. 3.

FIG. 5 illustrates a view along line V-V of FIG. 4.

FIG. 6 illustrates a modification to a system such as illustrated in FIG. 1, in use within a body lumen of a patient.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

In accordance with various principles of the present disclosure, devices, systems, and methods are disclosed herein for isolating a working area within a patient's body. It will be appreciated that terms such as working area or working space or working zone or working region or working chamber (and similar terms) are usable interchangeably herein without intent to limit. The working area generally is an area of a patient's body at which a procedure is to be performed and/or in which a target site is located. The target site may be the target of the procedure to be performed (e.g., may be a lesion to be removed and/or biopsied), and may alternately be referenced herein as a target location, treatment site, target tissue, target tissue site, deployment site, etc., including combinations thereof and other grammatical forms thereof, without intent to limit. In some aspects, it is desirable to isolate the working area and/or target site from surrounding areas, such as to isolate the working area and/or target site from environmental influences outside the working area and/or target area. Environmental influences include, without limitation, anatomical structures, materials, substances, etc., extraneous to the working area and target site and which do not need to be or should not be in the working area or at the target site in order to perform, or during performance of, a procedure. It will be appreciated that terms such as isolate, separate, segregate, confine, enclose, close, block, seal, cover, etc. (including other grammatical forms thereof) are usable interchangeably herein without intent to limit. In some aspects, isolation of a target site may be beneficial in reducing, and even eliminating, interference by surrounding anatomical structures, materials, substances, etc., and/or processes, which typically occur in the working area. For instance, such isolation may serve as a solution to the problem of incomplete colonoscopy preparation of the bowel prior to an endoscopic procedure. Devices, systems, and methods of the present disclosure may be used to allow a medical professional to clean the working area along a target area in the bowel with respect to which a procedure is performed, without necessarily cleaning the entire bowel. By cleaning the bowel area (or, more generally, addressing a process as limited to the working area and target site) and wall (or, more generally, the target site, without also addressing surrounding areas outside the working area and target site) to a sufficient level at the working area and target site, the medical professional may proceed with performing the procedure without the need for addressing incomplete bowel preparation in other areas not to be accessed or affected (at least directly) during the procedure. Thus, by isolating the target area in the bowel from the surrounding areas which contain fecal matter, the medical professional can clean only the necessary working area, without worrying about fecal matter surrounding areas and/or ongoing processes in the gastrointestinal tract (e.g., natural movement of materials which may cause the working area to become dirty again if not isolated).

In some aspects, devices and systems of the present disclosure include a chamber with one or more openings through which a target site may be accessed from within and/or through the chamber. In accordance with various principles of the present disclosure, a proximal end, a distal end, or both the proximal end and the distal end of the chamber are closed to isolate the space therebetween, such as from proximal and distal environmental influences. For instance, a covering, membrane, wall, seal, enclosure, and/or other material may be provided along or over one or both ends of the chamber to isolate the proximal and/or distal ends of the chamber from regions of the body proximal and/or distal to the chamber. A substantially closed working space is thereby created by the chamber for performing a procedure without influence or interference of surrounding areas of the body. In some aspects, an expandable (e.g., radially-expandable) member forms the chamber, and a covering is provided over one or both ends of the expandable member to isolate the chamber space between the ends thereof.

In some aspects, systems of the present disclosure include one or more medical instruments, tools, devices, etc. (such terms being usable interchangeably herein without intent to limit) having a working end configured for use in performing a procedure with respect to the target area. In some aspects, devices and systems of the present disclosure are used with a delivery device with which the one or more medical instruments may be delivered, such as a catheter, sheath, overtube, medical scope (e.g., endoscope, arthroscope, bronchoscope, colonoscope, cystoscope, duodenoscope, gastroscope, hysteroscope, laparoscope, ureteroscope, etc.), etc. In some aspects, the medical device is a medical scope. In some aspects, the system further comprises a further medical device deliverable to the target area with the medical scope, such as through a working channel of the medical scope. In some aspects, the medical scope and the further medical device may collectively be considered a medical device.

In some aspects, a chamber device formed in accordance with various principles of the present disclosure is inserted separately and independently (e.g., before, after, alongside, but not through) a medical instrument of a system of the present disclosure. In some aspects, a chamber device is operatively associated with a medical instrument or delivery device, such as a medical scope, such as positioned over a distal end of the medical scope, such as to facilitate use of the medical scope (e.g., a medical instrument delivered by the medical scope) within and through the chamber device.

In some aspects, devices, systems, and method of the present disclosure may be used in minimally-invasive procedures, e.g., transluminal, transcatheter, endoscopic, etc., procedures which do not require open surgery (cutting open the patient), but, instead, access a target site within the patient via a natural orifice (or, in some instances, a small incision not considered to constitute an open-surgery cut). As such, the device and system may be sufficiently flexible to navigate through tortuous body passages to access a treatment site within a patient remote from the medical professional.

Various embodiments of devices, systems, and methods for creating a working space within a body will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that, in the figures of the accompanying drawings, common features are identified by common reference elements and, for the sake of brevity and convenience, and without intent to limit, the descriptions of the common features are generally not repeated. For purposes of clarity, not all elements or features having the same reference number are numbered. Moreover, a group of similar elements may be indicated by a number and letter, and reference may be made generally to one or such elements or such elements as a group by the number alone (without including the letters associated with each similar element).

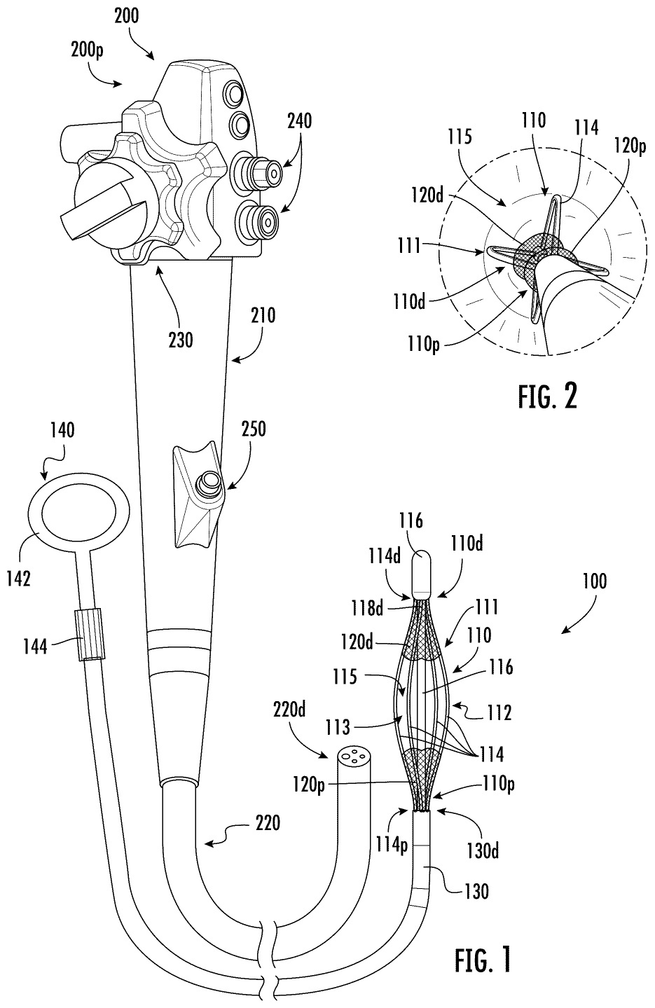

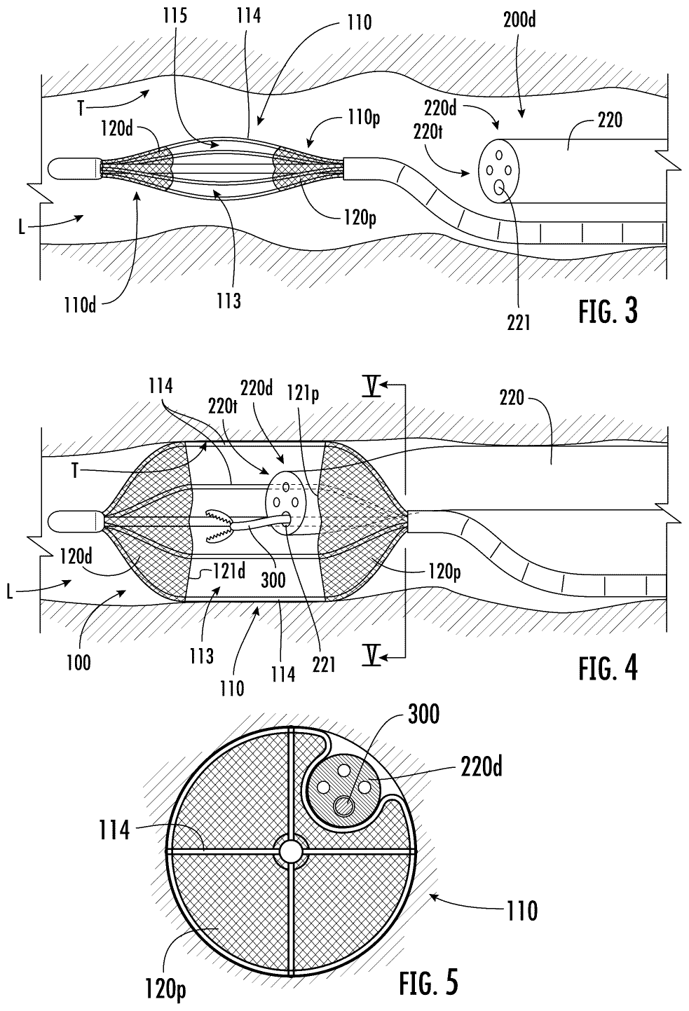

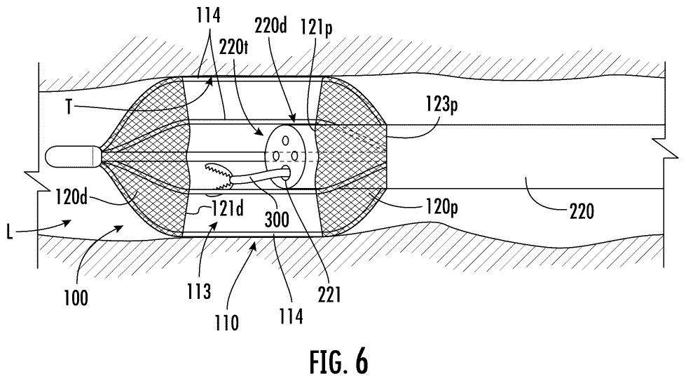

Turning now to the drawings, an example of an embodiment of a chamber device 100 configured to create a working space within a body is illustrated in FIG. 1 along a medical device 200. The chamber device 100 includes a chamber member 110 having a proximal end 110p and a distal end 110d and defining a chamber 113 therein (e.g., defined by a chamber wall 111). In the illustrated example of an embodiment, the chamber 113 extends between the proximal end 110p and the distal end 110d of the chamber member 110. In some aspects, the exterior of the chamber member 110 contacts an anatomical structure at or along which a target area T is located (such as illustrated in FIG. 3, FIG. 4, and FIG. 6, and described in further detail below). The chamber member 110 is formed with one or more openings 115 communicating the chamber 113 therein with the patient's anatomy (e.g., an anatomical wall or other anatomical structure) outside/external to the chamber member 110. As such, a working area is created by the chamber member 110 (e.g., by the chamber 113) along or adjacent the target area T so that medical devices and/or instruments may be extended into the working area as well as through the chamber wall 111 to access the target area T, and used to perform a procedure with respect to the target area T (as described in further detail below).

In accordance with various principles of the present disclosure, the working space defined within the interior of the chamber member 110 communicates (e.g., fluidly or otherwise) with anatomical structure along or adjacent the exterior of the chamber member 110 via the one or more openings 115 through the chamber wall 111. In the example of an embodiment illustrated in FIG. 1, the chamber wall 111 is defined by a plurality of elongate elements 114. At least two adjacent elongate elements 114 are spaced apart from each other to define an opening 115 through the chamber wall 111 to communicate the chamber 113 with at least a portion of the patient's anatomy outside the chamber wall 111 (e.g., along the exterior of the chamber wall 111, with the chamber 113 being defined by the interior of the chamber wall 111), as may be appreciated with reference to the proximal view of the chamber member 110 illustrated in FIG. 2. The remaining elongate elements 114 may include additional adjacent elongate elements 114 spaced apart from each other to form additional openings. Additionally or alternatively, two or more elongate elements 114 may be adjacent one another to form a continuous wall, the present disclosure not being limited in this regard. The elongate elements 114 may be wires, bands, strips, struts, filaments, etc., with sufficient rigidity to maintain a desired shape or configuration to define the chamber 113 therein. In some aspects, the elongate elements 114 are sufficiently rigid to shift or move from a delivery configuration (e.g., a compact configuration, such as generally elongated and substantially straight) to an expanded configuration in which the elongate elements 114 are spaced radially apart from one another (e.g., bowed, flexed, bent, curved, etc., away from one another) to form (and maintain a desired shape or configuration of) the chamber 113. In some aspects, the elongate elements 114 are formed of a flexible material such as nitinol, stainless steel (e.g., medical grade stainless steel), a polymeric or plastic material, or other material capable of maintaining a shape independently of another supporting structure (e.g., having sufficient rigidity to define a desired configuration or position or shape independently of another structure). In some aspects, the elongate elements 114 are nonetheless sufficiently flexible to conform to the anatomical structure along which the elongate elements 114 are positioned. It will be appreciated that although the elongate elements 114 are illustrated as extending generally longitudinally and generally parallel to one another, other configurations are within the scope and spirit of the present disclosure. For instance, a braided wall of elongate elements 114, spirally extending elongate elements 114, etc., may be provided instead of longitudinally extending elongate elements 114, the present disclosure not being limited in this regard.

Further in accordance with various principles of the present disclosure, the chamber device 100 includes one or more sealing members 120 operatively associated with the chamber 113 to seal a working space defined by the chamber 113 with respect to the target area in the patient's body. The one or more sealing members 120 seal the chamber 113 from other areas and/or structures of the anatomy and/or other (e.g., extraneous) materials and/or substances in the region of (e.g., surrounding) a target area within the patient, and/or processes occurring elsewhere but which are not desired, or are contraindicated, in the working area in the vicinity of/adjacent the target area. It will be appreciated that references to structures, materials, substances, processes, etc., from which the working area is to be sealed may be referenced in various interchangeable manners herein with the same or similar intent throughout the present disclosure and without detracting or limiting the present disclosure unless explicitly stated, those of ordinary skill in the art being capable of understanding the type of isolation desired for a given procedure. The one or more sealing members 120 are configured to isolate, seal, block, close, enclose, etc., an area on one side of a sealing member 120 from an area on the other side of the sealing member 120. In other words, the sealing members 120 form a barrier, seal, etc., such as a fluid impermeable wall, isolating the working area from other areas within the patient's body. The working area created by the chamber member 110 may thus be isolated or sealed off or otherwise unaffected by surrounding areas and/or materials. The chamber device 100 thereby forms a working area within the chamber member 110, communicated with a target area of the patient's body through one or more openings through the chamber wall 111, and isolated from areas of and/or substances in the patient's body which are not in the working area or target area. A medical professional may thus perform a procedure on or with respect to the target area with instruments positioned in the working area created by a chamber device 100 formed in accordance with various principles of the present disclosure, and without influence, interference, etc., by structures, substances, etc., not associated with the target area. For example, as described in further detail below, a section of an anatomical structure such as the bowel may be isolated from proximal and distal areas within the bowel. More particularly, if the bowel has not been sufficiently cleared to perform a procedure at the working area, a chamber device 100 formed in accordance with various principles of the present disclosure may be used to isolate the working area, and clear the area of matter interfering with performance of the procedure, without needing to clear the entire bowel of such matter.

In some aspects, the elongate elements 114 may be considered to form a support or scaffold or cage on which the one or more sealing members 120 may be disposed (e.g., formed, positioned, covered, etc.). The one or more sealing members 120 may be flexible covering member such as a membrane or other material capable of forming the desired seal. The one or more sealing members 120 may be formed as a separate layer of material disposed with respect to (e.g., over, within, etc.) the chamber wall of the chamber member 110, and/or may be formed on (e.g., molded, dipped, spray coated over a mandrel, etc.) the elongate elements 114, or otherwise operatively associated with the elongate elements 114, the present disclosure not being limited in this regard. If a sealing member 120 is formed separately from the elongate elements 114 forming the chamber wall 111, the sealing member 120 may be coupled to one or more of the elongate elements 114 with the use of a crimping ring or other coupling element known to those of ordinary skill in the art. Additionally or alternatively, the sealing member 120 may be bonded, welded, adhered, or otherwise coupled with the chamber member 110.

In some aspects, the chamber wall 111 of the chamber member 110 is configured to be sufficiently flexible to substantially follow or match or otherwise conform to the contour of the anatomical structure at or along which the target area is located. Additionally or alternatively, in some aspects, the one or more sealing members 120 are operatively associated and sized, shaped, configured, dimensioned, and/or arranged (such as with respect to the chamber wall) to contact the anatomical structure at or along which the target area is located to seal a working area defined within the chamber member 110 with respect to such anatomical structure. Additionally or alternatively, in some aspects, the one or more sealing members 120 are sufficiently flexible to substantially follow or match or otherwise conform to the contour of the anatomical structure at or along which the target area is located. It will be appreciated that the chamber member 110 (e.g., the chamber wall 111 forming the chamber member 110) may be configured (e.g., shaped, contoured, dimensioned, etc.) such that a single sealing member 120 suffices to enclose a working area defined by the chamber member 110 and to seal the working area and the anatomical target area from other anatomical structures or substances outside the chamber device 100.

The example of an embodiment of a chamber device 100 illustrated in FIG. 3, FIG. 4, FIG. 5, and FIG. 6 is configured for insertion into a body lumen L, and to extend within a body lumen L, for performance of a procedure within/with respect to the body lumen L. The chamber 113 of the chamber device 100 is thus configured to define a working area adjacent and in communication with a target area T of the body lumen L (indicated in the accompanying figures generally as towards one region along the internal circumference of the body lumen L, but which may extend circumferentially around the body lumen L). In accordance with various principles of the present disclosure, one or more sealing members 120 are operatively associated with the chamber wall to seal the chamber 113 from areas of the anatomy and/or substances proximal and distal to the working space created by the chamber 113 and the target area T of the body lumen L. More particularly, a proximal sealing member 120p is operatively associated with the proximal end 110p of the chamber member 110 to seal the proximal end 110p of the chamber member 110, and a distal sealing member 120d is operatively associated with the distal end 110d of the chamber member 110 to seal the distal end 110d of the chamber member 110.

The chamber 113 defined by the chamber member 110 is thus operatively in communication with the target area T (e.g., via the openings 115), forming a working area sealed by the sealing members 120 against communication with areas and/or substances surrounding and/or outside of the working area. In some aspects, as may be appreciated with reference to FIG. 4 and FIG. 6, the proximal sealing member 120p has a peripheral edge 121p (e.g., distal to the proximal end 100p of the chamber member 110) in sealing engagement with the anatomical structure (e.g., at or along which the target area T is located) to seal the proximal end 100p of the chamber member 110 along and/or with respect to an internally facing side of the proximal sealing member 120p. Additionally or alternatively, the distal sealing member 120d has a peripheral edge 121d (e.g., proximal to the distal end 100d of the chamber member 110) in sealing engagement with the anatomical structure (e.g., at or along which the target area T is located) to seal the distal end 100d of the chamber member 110 along and/or with respect to an internally facing side of the distal sealing member 120d. The one or more openings 115 through the chamber member 110 thus limit communication of the chamber 113 with the target site TS to within the confines of the isolated working area sealed with respect to the target area T by the sealing members 120. A working area is thereby defined by an interior of the chamber member 110 (e.g., an interior surface of the chamber wall 111) and interior sides (facing and on the same side as the chamber 113) of the sealing members 120. The working area seals the target area T with respect to structures and/or substances.

In the illustrated example of an embodiment, the chamber device 100 further includes a flexible elongate member 130 having a distal end 130d from which the chamber member 110 distally extends. In some aspects, the flexible elongate member 130 includes a lumen in which the chamber device 100 is positioned (fully or at least partially) for delivery to the target area T in a compact configuration. The compact configuration may be a collapsed, compressed, or otherwise smaller configuration than a deployed configuration to facilitate delivery thereof within a patient's body. It will be appreciated that the chamber member 110 may, additionally or alternatively, be configured to be delivered distal to the flexible elongate member 130 (not within a lumen of the flexible elongate member 130) in a compact delivery configuration and shiftable to an expanded deployed configuration. In the example of an embodiment illustrated in FIG. 1, the distal ends 114d of the elongate elements 114 are coupled to a distal member 116 at the distal end 110d of the chamber member 110 and the proximal ends 114p are maintained in close proximity (e.g., with a retaining ring or other manner such as known to those of ordinary skill in the art, the present disclosure not being limited in this regard). As may be appreciated, such arrangement of the ends 114p, 114d of the elongate elements 114 may facilitate formation of the desired shape or configuration of the chamber 113. Additionally, in the example of an embodiment illustrated in FIG. 1, an elongated controller 118 extends along and is axially translatable with respect to the elongate elements 114. The distal end 118d of the elongated controller 118 is coupled to the distal member 116, and the elongated controller 118 may extend proximally along (e.g., through a lumen defined in) the flexible elongate member 130 to a control handle 140. The proximal end of the elongated controller 118 is operatively associated with the control handle 140 so that operation of the control handle 140 axially translates (e.g., distally advance or proximally retract) the elongated controller 118 to move the distal member 116 (and thus the distal ends 114d of the elongate elements 114) with respect to the proximal ends 114p of the elongate elements 114 to cause the elongate elements 114 to move radially away from one another to radially expand the chamber member 110. The control handle 140 may include a first member 142 (e.g., a thumb ring) movable with respect to a second member 144 (e.g., a slide) to move the elongated controller 118 and the distal member 116 with respect to proximal ends 114p of the elongate elements 114. For instance, the first member 142 may be operatively associated with proximal ends 114p of the elongate elements 114, and the second member 144 may be operatively associated with the elongated controller 118 and the distal ends 114d of the elongate elements 114, or vice versa, so that movement of the first member 142 with respect to the second member 144 causes movement of the elongated controller 118 and the distal member 116 to move the ends 114p, 114d of the elongate elements 114 with respect to one another (e.g., closer together to expand the chamber wall 111 and further apart to collapse the chamber wall 111). In some aspects, the proximal ends 114p of the elongate elements 114 may extend proximally to be coupled with the first member 142, and/or may be coupled to the distal end 130d of the flexible elongate member 130, to move with movement of the first member 142. The elongated controller 118 would thus be operatively coupled to move with movement of the second member 144. Other arrangements and configurations such as known to those of ordinary skill in the art are within the scope of the present disclosure, the present disclosure not being limited in this regard.

In accordance with various principles of the present disclosure, a system for creating a working space within a body includes the chamber device 100 and a medical device 200, such as illustrated in FIG. 1. In some aspects, the medical device 200 has a flexible elongate member with an end effector or other operable element extending from or along the distal end of the flexible elongate member and configured for use in performing a procedure with respect to the target area. In some aspects, the medical device 200 is deliverable through a tubular flexible elongate member, such as a delivery catheter, shaft, tube, etc. In some aspects, the medical device 200 is a device configured to deliver one or more medical instruments configured to perform a procedure with respect to the target area T. In some aspects, reference to medical device 200 may include a medical scope as well as one or more medical instruments delivered therewith (e.g., through a working channel thereof).

The example of an embodiment of a medical device 200 illustrated in FIG. 1 is a medical scope 200 having a control handle 210 along a proximal end 200p thereof, and a flexible tubular elongate member 220 (also known as a tubular shaft or insertion member of the medical scope) extending distally from the control handle 210 and insertable into a patient in a manner known to those of ordinary skill in the art. The control handle 210 may include one or more controllers (e.g., knobs 230, such as for steering the flexible tubular elongate member 220); ports (e.g., ports 240, such as for fluidly coupling with a suction/vacuum source, a fluid source, etc.); and/or an insertion port 250 through which a medical device may be extended into and through the flexible tubular elongate member 220); and/or other connections (e.g., electrical, light, visualization such as with a camera, etc.). The flexible tubular elongate member 220 has a distal end 220d which is insertable into a patient to be advanced to a target area T. The flexible tubular elongate member 220 defines one or more lumens or working channels 221 through which one or more medical tools 300 may be extended and delivered to a target area T, such as illustrated in FIG. 3, FIG. 4, and FIG. 6. The distal tip 220t of the flexible tubular elongate member 220 has an end face with various features for use of the medical device 200 in performing a medical procedure. For instance, a working channel 221 defined through the flexible tubular elongate member 220 may open at the distal tip 220t of the flexible tubular elongate member 220 for delivery of a medical instrument 300 to the target area T. Other optional features are schematically illustrated but not called out with reference numbers because the configurations and arrangements may vary from medical scope to medical scope, without affecting or otherwise limiting the present disclosure. The optional features may include, without limitation, a visualization element (e.g., a camera lens, optic fiber, imager, etc.); a fluid supply port (such as to supply irrigation fluid to the target area T; and/or to supply fluid, such as gas, to insufflate the interior of the patient at the target area T; and/or to supply a gas/lens wash nozzle to wash or otherwise clear or clean a lens covering the imager); a suction port (with suction additionally or alternatively being applied through the working channel 221); one or more illumination windows that convey illumination light to the target area T; and/or other features known to those of ordinary skill in the art. It will be appreciated that other suitable configurations of a medical scope 200 may be utilized than the medical scope 200 illustrated in FIG. 1 and discussed herein.

In accordance with various principles of the present disclosure, as described above, the chamber device 100 forms a working area in which the distal end 200d of the medical device 200 (e.g., the distal end 220d of the flexible tubular elongate member 220) may be extended, so that one or more medical instruments may be delivered, by the medical device 200, into the working area and used to perform a procedure with respect to the target area T. The flexible tubular elongate member 220 is illustrated in FIG. 3 as being advanced within the body lumen L to the target area T so that the distal end 220d approaches the proximal end 100p of the chamber device 100. The chamber device 100 is optionally expandable, such as described above. In some aspects, the chamber device 100 may be in a delivery configuration as the distal end 220d of the flexible tubular elongate member 220 is advanced towards the chamber device 100, and expanded after the distal end 220d of the flexible tubular elongate member 220 has approached the proximal end 100p of the chamber device 100. In some aspects, the distal end 220d of the flexible tubular elongate member 220 is advanced to extend the distal tip 220t thereof to be positioned between the proximal sealing member 120p and the distal sealing member 120d and longitudinally along the area of the chamber device 100 which forms the working area adjacent a target site at which a procedure is to be performed. In some aspects, if only a single opening 115 is formed along the chamber wall 111, it would be optimal to position the distal tip 220t adjacent the single opening 115 so that the distal tip 220t extends into the chamber 113 once the chamber device 100 is expanded to define the chamber 113. In some aspects, if the chamber wall 111 is formed from a plurality of elongate elements 114, it may be advantageous to leave openings 115 between more than one pair of adjacent elongate elements 114 to facilitate positioning of the distal tip 220t through the chamber wall 111 to be positioned in the chamber 113. Alternatively, the chamber device 100 may be expanded, and then the distal tip 220t of the flexible tubular elongate member 220 may be distally advanced past the proximal sealing member 120p and into an opening 115 through the chamber wall 111 and into the working space defined by the chamber 113.

As may be appreciated with reference to FIG. 4, the sealing members 120 seal against the patient's anatomy to sealingly enclose the working space. Once the chamber 113 defines a working space adjacent the target area T, and the distal tip 220t is positioned within the working space, such as illustrated in FIG. 4, a medical instrument 300 may be advanced out of the working channel 221 of the flexible tubular elongate member 220 to be used to perform a procedure with respect to the target area T. Suction may be applied (e.g., via the working channel 221 and/or a dedicated suction port in the distal tip 220t) to clear the working area of debris from the working area. For instance, in the case of a gastrointestinal procedure, the working area in the patient's bowel may be cleared of fecal matter without the need to clear upstream (distal) or downstream (proximal) regions of the bowel, facilitating the procedure, minimizing procedure time, and, in some cases, even making the procedure performable under circumstances which would otherwise require rescheduling of the procedure.

As may be appreciated from the proximal end view of FIG. 5, along line V-V of FIG. 4, the arrangement of the chamber device 100 and medical device 200 of the system illustrated in FIG. 3 and FIG. 4 results in deformation (e.g., radially-inward compression) of the proximal sealing member 120p along the longitudinal extent along which the distal end 220d of the flexible tubular elongate member 220 extends to advance the distal tip 220t thereof into the chamber 113. Preferably, the proximal sealing member 120p is sufficiently flexible and/or elastic to maintain the seal with respect to the body lumen L with the medical device 200 extended therealong as illustrated. An alternative embodiment of a system formed in accordance with various principles of the present disclosure is illustrated in FIG. 6 in which the proximal sealing member 120p may be formed/positioned around the external periphery of the distal end 220d of the flexible tubular elongate member 220, proximal to the distal tip 220t. More particularly, the proximal sealing member 120p may be sealed with respect to the distal end 220d of the flexible tubular elongate member 220 so that the proximal end of the chamber 113 is sealed by a proximal peripheral edge 123p of the proximal sealing member 120p bonded, adhered, or otherwise sealed with respect to the exterior surface of the distal end 220d of the flexible tubular elongate member 220. As may be appreciated, the chamber device 100 may be mounted on the distal end 220d of the flexible tubular elongate member 220 to be delivered with the distal end 220d of the flexible tubular elongate member 220 to the target area T.

It will be appreciated that all apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

Although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like. Various further benefits of the various aspects, features, components, and structures of devices, system, and methods such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and / or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A medical device configured to create a working space along a target area within a body of a patient, said device comprising:
a chamber device including a chamber member with a proximal end and a distal end and defining a chamber therein between the proximal end and the distal end and defining a working space in communication with the target area; and
at least one sealing member operatively associated with the chamber to seal the working space from surrounding patient anatomy, materials, and/or substances.

2. The device of claim 1, wherein said at least one sealing member includes a proximal sealing member sealing the proximal end of the chamber member, and a distal sealing member sealing the distal end of the chamber member.

3. The device of claim 2, wherein:
said chamber device comprises a chamber wall defining the chamber and sized to fit within a body lumen within a patient to contact the interior of the body lumen;
said proximal sealing member seals the proximal end of the chamber member with respect to the body lumen;
said distal sealing member seals the distal end of the chamber member with respect to the body lumen; and
at least one opening is defined through said chamber wall to communicate the chamber with the target area while the working space remains sealed from surrounding patient anatomy, materials, and/or substances.

4. The device of claim 1, wherein said chamber device comprises a chamber wall defining the chamber and formed of a plurality of elongate elements, wherein at least two adjacent elongate elements are spaced apart to define an opening through said chamber wall to communicate the chamber with the target area.

5. The device of claim 4, wherein said elongate elements are flexible to conform an exterior of said chamber wall to the target area with said at least one sealing member sealing against the target area.

6. The device of claim 4, wherein said elongate elements are flexible elongate elements are shiftable from a compact delivery configuration to an expanded configuration defining the chamber.

7. The device of claim 6, further comprising:
an elongate member having a distal end and a proximal end, said chamber device extending from a distal end of said elongate member; and
a control handle operatively coupled with said plurality of elongate elements to shift said plurality of elongate elements between the delivery configuration and the expanded configuration.

8. The device of claim 4, wherein said at least one sealing member covers spaces between adjacent elongate elements to form a barrier between the chamber and the exterior of the chamber device.

9. The device of claim 1, wherein said at least one sealing member comprises a membrane.

10. The device of claim 1, wherein said at least one sealing member includes a sealing member which is flexible to allow a medical device to be passed between the flexible sealing member and the target area and to maintain a seal with respect to the target area.

11. A system for creating a working space along a target area within a body of a patient, said system comprising:
a chamber device comprising:
a chamber member with a proximal end and a distal end and defining a chamber therein between the proximal end and the distal end and in communication with the target area; and
at least one sealing member forming a barrier sealing the chamber with respect to the target area to create a working space sealed from surrounding patient anatomy, materials, and/or substances; and
a medical device having a distal end configured for use in performing a procedure with respect to the target area.

12. The system of claim 11, wherein said medical device includes a delivery device with a lumen therethrough and an additional medical device extendable through the lumen.

13. The system of claim 12, wherein said medical device is a medical scope.

14. The system of claim 11, wherein said medical device is extendable along said at least one sealing member and into the chamber.

15. The system of claim 11, wherein said at least one sealing member includes a proximal sealing member sealed around the exterior of a distal end of said medical device with the distal end of said medical device extending into the chamber.

16. A method of creating a working space along a target area within a body of a patient, said method comprising:
advancing a chamber device to the target area, the chamber device defining a chamber;
sealing a sealing member provided over the chamber device with respect to the target area to seal the chamber with respect to the target area and from surrounding patient anatomy, materials, and/or substances; and
communicating the chamber with the target area via one or more openings defined through the chamber device and sealed with respect to the target area and from surrounding patient anatomy, materials, and/or substances.

17. The method of claim 16, further comprising:
sealing a proximal sealing member provided over a proximal end of the chamber device with
respect to the target area to seal a proximal end of the chamber with respect to the target area and from proximal surrounding patient anatomy, materials, and/or substances; and
sealing a distal sealing member provided over a distal end of the chamber device with respect to the target area to seal a distal end of the chamber with respect to the target area and from distal surrounding patient anatomy, materials, and/or substances.

18. The method of claim 16, further comprising advancing the chamber device into a body lumen and sealing the chamber device with respect to the body lumen to seal the chamber with respect to portions of the body lumen proximal and distal to the target area.

19. The method of claim 18, further comprising extending a distal end of the medical device past a proximal sealing member and into the chamber.

20. The method of claim 18, wherein the medical device comprises a medical scope and a proximal sealing member seals about the exterior of a distal end of the medical scope to seal the chamber with respect to the distal end of the medical scope, said method further comprising advancing a further medical device through a working channel of the medical scope and into the chamber defined by the chamber device.
